(19) United States
(12) Patent Application Publication
(10) Pub. No.: US 2006/0246526 A1
(43) Pub. Date: Nov. 2, 2006

(54) Microfluidic affinity assays with improved performance

(75) Inventors: Mats Inganas, Uppsala (SE); Magnus Ljungstrom, Uppsala (SE)

Correspondence Address: FULBRIGHT & JAWORSKI, LLP, 1301 MCKINNEY, SUITE 5100, HOUSTON, TX, 77010-3095, US

(73) Assignee: Gyros Patent AB, Uppsala (SE)

(21) Appl. No.: 11/292,996

(22) Filed: Dec. 2, 2005

Related U.S. Application Data
(63) Continuation of application No. PCT/SE04/00844, filed on Jun. 2, 2004.
(60) Provisional application No. 60/475,125, filed on Jun. 2, 2003.

(30) Foreign Application Priority Data
Jun. 2, 2003 (SE) 0301616-9

(51) Int. Cl. G01N 33/53

(52) U.S. Cl. 435/7.93

(57) ABSTRACT

Method for measuring the signal from a label in a labeled measuring reagent that has been specifically adsorbed to its affinity counterpart in a zone of a porous nanolitre (nl) bed that is present in a microchannel structure of a microfluidic device. The measuring is part of an assay for determining an analyte of a sample by performing one, two or more heterogeneous specific affinity reactions which comprises that said labeled measuring reagent becomes affinity bound to said zone.

### CROSS-REFERENCE TO RELATED APPLICATIONS

This application is a continuation of application PCT/SE2004/000844 filed on Jun. 2, 2004 which claims priority to U.S. Provisional Application No. 60/475,125 filed Jun. 2, 2003 and Swedish Application No. 0301616-9 filed Jun. 2, 2003.

### TECHNICAL FIELD

The present invention relates to a method for measuring a labeled measuring reagent that has been selectively adsorbed to its immobilized affinity counterpart in a zone of a porous nanolitre bed (nl-bed) that is present in a microchannel structure of a microfluidic device. The method may be used in assays for determining an analyte by performing one, two or more heterogeneous specific affinity reactions, one of which comprises the selective adsorption of the labeled measuring reagent to its affinity counterpart. The amount of labeled measuring reagent that is bound to the zone by the adsorption is a function of the amount of the analyte in the sample assayed.

### BACKGROUND OF THE INVENTION

Microfluidic devices became of interest during the early nineties and were then primarily intended for performing large numbers of capillary electrophoresis experiments in parallel. It was early recognized that microfluidic devices also could be used for performing affinity assays for characterization of reaction variables of analytes (e.g. amounts). These affinity assays typically utilized heterogeneous affinity reactions with labeled reactants, i.e. reactants equipped with analytically detectable groups. Typical tags or labels were radioisotopes, fluorophores, chemiluminophores, chromophors, enzymatically active and other catalytically active components (e.g. enzymes, substrates, co-substrates, cofactors, coenzymes etc), metal particles and metal ions, particles, affinity groups etc. However, severe sensitivity problems were encountered when the present inventors started to design ultra-sensitive assays for microfluidic devices intended for parallel processing of analytes and reagents. Many of the problems were of the same kinds as documented for larger samples but now became more pronounced and more difficult to handle. Typical problems dealt with: 1) significant non-specific signals emanating from unwanted binding of reactants to the solid phase, 2) disturbances from the solid phase as such and/or from the material keeping the solid phase in place etc. The risk for low sensitivity and/or unacceptable inter- and intra-device variations initially turned out to be unacceptably high for microfluidic devices.

An important step forward was accomplished when the present inventors recognized the importance of carrying out heterogeneous affinity reactions under flow conditions and under common flow control. See for instance WO 02075312 (Gyros AB). Another important step was the recognition that the creation of various kinds of background images of the nl-volumes or beds containing the solid phase was a powerful technique for removing disturbances (noise) in a raw data image of these volumes/beds. The result enabled an increased sensitivity for various microfluidic affinity assays and other bioassays. See for instance WO 03025548 (Gyros AB) and WO 0356517 (Gyros AB) and corresponding U.S. application Ser. No. 10/331,399. The labels suggested in these patent applications include enzymes. Theses principles are potentially also applicable to the catalytic assays described in WO 03098302 (Gyros AB)

It would be advantageous to have access to alternative sensitivity-increasing methods and means for use alone or in combination with known alternatives for heterogeneous affinity assays in microfluidic devices.

### BRIEF SUMMARY OF THE INVENTION

The main objects of the invention are to provide methods and/or means that will increase sensitivity of affinity assays of the kind given above, i.e. of assays in which the concentration of the analyte in the sample is in the nM-range, i.e. ≦5,000×10⁻⁹ M, such as in the picomolar (pM-range) i.e. ≦5,000×10⁻¹² M or ≦1,000×10⁻¹² M or ≦100×10⁻¹² M or even lower, such as in the femtomolar range (fM-range), i.e. ≦5,000×10⁻¹⁵ M, such as ≦1,000×10⁻¹⁵ M. These objects also include providing complete assaying methods that have the increased sensitivity.

A subobject is that the methods and/or means shall be capable of lower the detection limit to be ≦10%, such as ≦1% or ≦0.1% of the detection limit for an analyte measured according to a particular assay protocol. The comparison shall be made in analogy with the comparison made in the experimental part.

### BRIEF DESCRIPTION OF THE DRAWINGS

FIG. 1 shows reference curves for the reference method and the innovative variant of the experimental part.

### DETAILED DESCRIPTION OF THE INVENTION

Patents and patent applications cited herein are hereby incorporated by reference.

Definitions

The expression “selectively adsorbed” and the like mean that the labeled measuring reagent is adsorbed to a higher extent within than outside the zone.

The term “heterogeneous affinity reaction” contemplates an affinity reaction between an affinity reactant that is present in a liquid and its counterpart that is prebound (=immobilized in a previous step by the user or manufacturer) to a solid phase, for instance a porous bed. After the reaction the liquid with unreacted reactant is separated from the solid phase. Depending on the further processing, the solid phase may subsequently be washed.

The expression “measuring an analyte” means that the amount and/or one or more properties of the analyte are measured. In the context of the invention the analyte is typically an affinity reactant. “Amount” refers to the presence or absence of an analyte in a sample, and may be measured in mass units, molar units, relative amounts, concentrations, activity/mass units etc. The expression “a property of an analyte that is an affinity reactant” and the like include affinity, i.e. affinity constants, rate constants for the formation and/or dissociation of affinity complexes etc. The expression “measuring an analyte” also includes that optimal reaction conditions for an affinity reaction between two affinity reactants are determined in which case one of the reactants, typically the reactant that is present in the lowest concentration, arbitrarily is designated to be the analyte. See further WO 02075312 (Gyros AB).

The term “analogues” is used for two or more affinity reactants that are capable of inhibiting or competing with each other for affinity binding to the same affinity counterpart and/or binding site. The term “analogue” is also used in the same manner for analytes.

The Invention

The present inventors have managed to overcome the problems discussed above by using labels that are components of catalytic systems. This was contrary to common knowledge in the field that says that amplification of signals from affinity reactants that are labeled with this kind of components will typically also include amplification of background signals, e.g. the signal related to non-specific binding.

The present inventors thus have recognized that the objects above can be accomplished provided:

a) the label on the measuring reagent is a component of a catalytic signal-producing system, and

b) this signal-producing system is capable of converting a substrate to an analytically detectable product that becomes immobilized selectively within the same zone of the porous bed as the labeled measuring reagent is present.

One aspect of the invention thus is a method for detecting/measuring a labeled measuring reagent as discussed in the first paragraph of “Technical Field”. The method comprises the characterizing features:

a) at least one of the heterogeneous affinity reactions comprises specifically adsorbing an analyte-related reactant to an excessive amount of its counterpart immobilized and homogeneously distributed within the porous nl-bed, and

b) the labeled measuring reagent comprises a label that is a component of a catalytic signal-producing system that converts a substrate to an immobilized analytically detectable product, and

c) the zone possibly contains one or more other components of the signal-producing system in immobilized form, and

d) the measuring comprises the steps of:

i) providing the components of the signal-producing system that are necessary for the formation of the immobilized product within the zone, and

ii) producing the immobilized product within zone.

Analyte-Related Reactants

An analyte-related reactant is a reactant that during an affinity assay becomes bound in an affinity complex such that its amount in the complex and/or not bound in the complex is a function of the amount of analyte in the sample. If the affinity reaction is heterogeneous there is typically a separation between reactants bound and not bound to the solid phase (porous bed).

The analyte-related reactant referred to in the characterizing feature (a) may be:

I) the labeled measuring reagent referred to in characterizing feature (b), or

II) the analyte, or

III) some other affinity reactant that is used in the assay in an amount and during conditions such that its presence in the affinity complex formed and/or not bound in the complex becomes a function of the presence of the analyte in the sample.

If the analyte-related reactant in characterizing feature (a) above is not according to I or II, it typically comprises two or more spatially separated affinity binding sites. One of the sites is for an affinity reaction that relates this reactant to the analyte and the other site is for an affinity reaction that relates the labeled measuring reagent to the analyte via the analyte-related reactant.

An analyte-related reactant may be a conjugate, but may also be a reactant inherently comprising the necessary two binding sites. In the case that it is a conjugate that doesn't comprise a component of a catalytic signal-producing system, it is typically a covalent conjugate between two different affinity moieties, for instance between an antibody active moiety or an antigen moiety as the first moiety, and biotin, a hapten etc as the second moiety. The second moiety may also be called a reporter group since it is used to relate the amount of analyte to the labeled measuring reagent.

A labeled measuring reagent is typically a conjugate between an affinity reactant and a component of a catalytic signal-producing system. The conjugate is typically covalent, i.e. the reactant and the component is linked to each other by bonds of covalent nature. Alternatively the molecule used as label may inherently comprise a suitable binding affinity to be used in an assaying protocol.

Affinity adsorption of an analyte-related reactant to an excessive amount of its affinity counterpart immobilized homogeneously to a porous bed may take place under static conditions, or more preferably under flow conditions. The excessive amount and/or flow conditions will support a high yield and a high reproducibility in the adsorption. In combination they will secure that essentially all of the analyte-related reactant can be bound in an upstream zone of the porous bed and assist in increasing sensitivity. The appropriate flow rate for accomplishing this depends on a number of factors, such as the affinity constant and rates of the affinity reaction between the analyte-related reactant in the through-passing liquid and its immobilized affinity counterpart, the volume and/or pore sizes of the porous bed, the diffusion constant of the affinity reactant in the through-passing liquid (and hence also its size) etc. Typically the flow rate through the nl-bed should give a residence time of ≧0.010 seconds such as ≧0.050 seconds or ≧0.1 seconds with an upper limit that typically is below 2 hours such as below 1 hour. Illustrative flow rates are within 0.01-100 nl/sec, typically 0.1-10 nl/sec. Residence time refers to the time it takes for a liquid aliquot to pass through the porous bed.

In the case the microfluidic device comprises two or more microchannel structures that are to be used for performing a number of assay runs in parallel, it becomes important also to have the proper flow control in order to avoid unacceptable inter-channel and inter-assay variation between different devices and within the same device. An acceptable flow control depends on a particular assay protocol, concentrations of reactants, their diffusion properties and reaction rates, etc, and typically utilizes pressure drop means in the microconduit linked to the outlet of the porous bed and/or common flow control as defined in WO 02075312 (Gyros AB) and WO 03024548 (Gyros AB). Sufficient flow control in most cases means that the intra-channel variation for residence time is within the mean residence time for the porous beds used in a device ±90%, such as ±75% or ±50% or ±25%.

According to a preferred variant of the invention, common flow control is accomplished by performing the assays in a microfluidic device in which the microchannel structures are designed for driving liquid flow by centrifugal force, i.e. by spinning the device, and/or by capillary force. Typically each microchannel structure then has an upstream part that is closer to the intended spin axis than a downstream part. See for instance WO 02075312 (Gyros AB) and WO 03018198 (Gyros AB).

According to another preferred variant suitable pressure drop means typically comprises that the microconduit that is linked to the outlet of the porous bed in each microchannel structure of a device is designed as a restriction micronduit that is capable leveling out the inter-channel variation in flow resistance within a device, for instance by creating a pressure drop that is larger than the total resistance to flow upstream this microconduit in each microchannel structure.

For a restriction microconduit this typically contemplates that its largest cross-sectional area is less than the largest cross-sectional area of the inlet microconduits of the microcavity containing the porous bed, with preference for ≦0.25, such as ≦0.10, of said largest cross-sectional area. Preferentially these ranges apply for ≧10%, such as ≧50%, of the length of a restriction microconduit, often with absolute preference for ≧90% up to the whole length of the restriction microconduit. Other kinds of pressure drop means are also possible in a restriction microconduit: the inner surface may be rougher than the inner surfaces upstream the porous bed, and/or the length of a the restriction microconduit may be greater, such as ≧5 times or ≧10 times, than the length of the shortest inlet microconduit, etc.

The pressure drop in a restriction microconduit is typically proportional to its length and inversely proportional to its hydraulic cross-sectional area. An increase in length of the restriction microconduit may thus compensate for an increase in its cross-sectional area and vice versa.

Further information about pressure drop means and restriction microconduits is given in WO 02075312 (Gyros AB) and WO 03025548 (Gyros AB).

Catalytic Signal-Producing System

The catalytic signal-producing systems that can be used in the invention are well-known in the field of affinity assays in which one or more of the components of a catalytic signal-producing system are immobilized, for instance as a consequence of the heterogeneous affinity reactions and/or directly immobilized to the matrix of a solid phase. See for instance U.S. Pat. No. 4,366,241 (Syva) and U.S. Pat. No. 4,391,904 (Syva). U.S. Pat. No. 5,196,306 (E.I. du Pont) gives a special variant in which the product formed is immobilized by reaction with the solid phase. Labels in the form of oligonuclotides that are used as templates for rolling-circle amplification, PCR and the like has been described (Schweitzer et al., Nature Biotechnology 20 (2002) 359-365; and Fredriksson et al., Nature Biotechnology 20 (2002) 473-477).

The catalytic signal-producing system used in the invention thus may comprise a single catalytic system in which only one catalyst is utilized. Alternatively it may comprise a linked catalytic system in which one, two or more catalysts are linked to each other and/or to one, two or more non-catalytic reactions such that the product from the action of an earlier catalyst or non-catalytic reaction in the system is the substrate of a subsequent catalyst or a subsequent non-catalytic reaction. Substrates and products having this kind of relationship will henceforth be called intermediates of the catalytic signal-producing system. These intermediates are not substrates/products of the catalytic signal-producing system in the context of the present invention. Typically it is assured that components of the signal-producing system that are neither created as intermediates nor are components of one of the individual catalytic systems of the complete signal-producing system are present in excessive amounts.

In preferred variants at least one, two or more, preferably all, of the individual catalytic systems of the catalytic signal-producing system is a biocatalytic system which is a system in which at least the catalyst as such have biopolymer structure.

Illustrative components of the catalytic signal-producing system are, starting substrates and ending products, catalysts (enzymes), cofactors, cocatalysts (co-enzymes), intermediates, co-substrates to intermediates, inhibitors, effectors, activators etc with the names applicable to enzyme systems being given within brackets. None, one, two or more of these components except the component that is used as label in the labeled measuring reagent may be immobilized within the porous nl-bed in advance to the assay. If present this kind of immobilized components may or may not be initially present in the zone of the porous bed in which the labeled measuring reagent is affinity enriched, and/or upstream this zone.

Each of the components given in the preceding paragraph may potentially be used as label in the labeled measuring reagent. Thus the catalytic component in the measuring reagent may be either a substrate or a non-substrate components. In the case the component is a substrate it typically is copied in large numbers by the catalytic system, for instance labels in the form of oligonucleotides may be amplified by PCR or rolling-circle amplification.

The catalytic signal-producing system typically comprise one, two or more enzyme systems selected from at least one of: 1) Oxidoreductases (dehydrogenases, oxidases etc), 2) Transferases, 3) Hydrolases (esterases, carbohydrases, proteases etc), 4) Lyases, 5) Isomerases, and 7) Ligases.

A component of a biocatalytic system may be natural or may have been produced synthetically or recombinantly. The component may exhibit amino acid structure, peptide structure, such as oligo- or polypeptide structure, nucleotide structure, such as oligo- or polynucleotide structure, carbohydrate structure such as oligo- or polysaccharide structure, lipid structure, steroid structure, hormone structure etc. Synthetic compounds, for instance deriving from combinatorial libraries, potentially mimicking natural variants of components of catalytic systems are included.

The component of the catalytic system that is used as a label is typically covalently attached to an appropriate affinity reactant that is selected according to the assay protocol used. A large number of methodologies for preparing covalent conjugates are well-known in the field and may, depending on the structure of the affinity reactant and the catalytic component, utilize a group selected amongst amino, carboxy, hydroxy, thiol, disulfide, aldehyde, keto etc on one or both of the two moieties that are to form the conjugate. These groups may by naturally present or may have been introduced synthetically. Alternatively the label as such is capable of acting as an affinity reactant that becomes part of the affinity complex form. No separate conjugation to an affinity reactant is then required.

Typically it is appropriate to include a spacer arm for joining two entities in a conjugate to be used in the invention, for instance providing a length in the range of 1-100 atoms, such as 1-50 atoms.

The end product of the catalytic signal-producing system may be insoluble under the conditions provided within the porous bed and therefore precipitate where it is formed, i.e. within the zone comprising the labeled measuring reagent. Alternatively the catalytic signal-producing system provides an immobilization step in which a soluble intermediate reacts with groups on the porous bed to thereby form an immobilized compound that is the analytically detectable product or is further processed by the catalytic system to the analytically detectable product. In the case of further processing it is preferred to select the catalytic system such that the density of analytically detectable product is substantially the same as or higher than the density of the immobilized compound in the zone in which immobilization has taken place. Density in this context refers to amount/volume unit of product/compound. Further processing in this context includes processing within the same zone as the labeled measuring reagent is present, or release and re-concentrating in a zone that has a center downstream to the center of this zone.

Immobilization by reaction with groups on the porous bed includes formation of a) covalent bonds between an intermediate of the catalytic signal-producing system, and b) adsorptive-like bonds. Covalent immobilization may be illustrated by the system utilized in Tyramide Signal Amplification Kits sold by Molecular Probes Inc (Oregon, USA) which utilizes a peroxidase system for creation of oxygen that will activate the ortho-position of phenol groups thereby enabling covalent linking of a phenol-containing fluorophor to tyrosine residues that are frequently occurring in reactants used in heterogeneous affinity assays, for instance as immobilized reactants. See also our experimental part. Adsorptive like bonds are typically of the same general kinds as those utilized for affinity reactions in heterogeneous affinity assays although it is important to select affinity reactants and catalytic system in such a manner that the there will be no disturbing interference, for instance cross-reactions. For more details see U.S. Pat. No. 5,196,306, U.S. Pat. No. 5,583,001 and U.S. Pat. No. 5,731,158.

The catalytic systems should be selected such that the immobilization reaction (including precipitation) becomes faster than the intermediate product diffuses out from the zone.

The analytically detectable product may be detectable as such, for instance by being signal-emitting, i.e. being capable of emitting radiation or interacting with irradiation. Thus the product may be radioactive, fluorescent, chemiluminescent, chromogenic, etc and/or absorb and/or reflect etc and/or scatter UV, IR and/or visible light. The final measurement is then typically carried out by measuring its radioactivity, fluorescence, chemiluminescence, colour, light adsorption, reflectance etc. Fluorescent products exhibiting delayed fluorescence in combination with measurements utilizing the time-resolved principle may be advantageous, for instance if liquid processing is taking place within the spinnable microfluidic devices described elsewhere in this specification.

In an alternative variant the analytically detectable product needs further processing before it is measured. This kind of further processing includes, e.g. transformation of the product to a compound that is possible to measure by its radiation (e.g. emitted UV, IR and/or visible light) or radiation interacting properties as discussed in the previous paragraph. This kind of further processing may take place in the same zone as where immobilization has taken place and/or include formation of the measurable compound in a zone downstream this zone, e.g. in the same or in a downstream porous bed.

The components of the catalytic signal-producing system that are necessary for producing the analytically detectable product within the zone containing the labeled measuring reagent may be provided in different ways. In a typical variant all of the components except the component(s) corresponding to the label and the possible intermediate(s) are introduced via one or more inlet ports after the labeled measuring reagent has been immobilized in the porous bed. If required the components may be mixed in distinct mixing structures within the device, or before being introduced into the device with the goal that all necessary components should be present simultaneously in the zone comprising labeled measuring reagent.

The immobilized analytically detectable product may be formed under static conditions or under flow conditions. Flow conditions include intermittent flow conditions, i.e. the flow is stopped for a predetermined period of time to allow reaction whereafter the flow is restarted to displace the used liquid with a fresh aliquot containing the necessary components. Flow conditions will assist in obtaining high amplification. Intermittent flow conditions will have the same effect but may in addition assist in keeping a soluble intermediate within the zone until it has been immobilized, i.e. retain the concentrating effect that possibly has been obtained by utilizing flow conditions and/or excessive amount of affinity counterpart during binding of an analyte-related reactant to the porous bed. See above.

Protocols of Heterogeneous Affinity Assays

Protocols that can be used in the present invention may be selected amongst those that are well known for the determination of an unknown amount of an analyte by a heterogeneous biospecific affinity assay. These protocols encompass that one or more affinity counterparts (anti-analytes) to the analyte are used for the formation of an affinity complex, the amount of which is related to the amount of the analyte in a sample. This relation/function is accomplished as is well known in the field by selecting the appropriate reaction conditions including amount of reactants. Depending on the protocol used this complex may or may not comprise the original analyte of the original sample introduced into the microfluidic device.

According to the protocols used in the present invention the affinity complex to be measured and related to the analyte comprises the above-mentioned labeled measuring reagent that has been selectively adsorbed within a zone of the porous bed. The labeled measuring reagent may be adsorbed to its immobilized affinity counterpart. Alternatively, the labeled measuring reagent is initially allowed to form a soluble affinity complex with a soluble form of its counterpart. Thereafter the complex is affinity adsorbed to an immobilized affinity reactant that is capable of binding to a site on the counterpart that is not interacting with the labeled measuring reagent.

Absorption may take place to an affinity reactant that a) is directly attached to the matrix of the porous bed, or b) is attached to the matrix via an affinity reactant that has been pre-immobilized to the bed, for instance by the manufacturer of the device. In the latter case a pre-immobilized affinity reactant in preferred variants of the invention is a general binder that will permit the customers to immobilize their own unique assay components, i.e. affinity reactants that are specifically adapted to what is going to be assayed. See for instance PCT/SE2004/000440.

Introduction of the analyte and other reactants that shall be related to the analyte may take place in sequence, in parallel, and/or as mixtures. One or more inlet ports of the device may be used. If needed, mixing of affinity reactants and liquids may take place within separate mixing units that are located upstream the porous bed. As discussed above at least one of the steps used comprises that an analyte-related reactant is captured by an excessive amount of its immobilized affinity counterpart. According to preferred embodiments of the invention, the reaction with an excessive amount can be carried out during diffusion-limiting or non-diffusion-limiting conditions. For a given system, the flow rate may in principle be used to secure that these conditions are at hand to obtain the largest possible concentration on the porous bed (smallest possible zone width), the general guide-line being that a decrease in flow rate (increase in residence time) will favor non-diffusion limiting conditions and vice versa for diffusion-limiting conditions. These rules primarily apply to large molecules.

There are in principle two general types of protocols: 1) competitive protocols that in the context of the invention include inhibition and displacement protocols, and b) non-competitive protocols. See also WO 02075312 (Gyros AB).

Competitive/Inhibition Protocols.

In these protocols the analyte and an analyte analogue are competing with each other for binding to a limiting amount of an anti-analyte. This anti-analyte may be a) immobilized or immobilizable if the analyte analogue is soluble and analytically detectable, and b) analytically detectable if the analyte analogue is immobilized or immobilizable.

Analytically detectable in this context means that the analyte analogue and the anti-analyte, respectively, may contain a natural affinity group or be a conjugate between an unconjugated form of the anti-analyte and either another affinity reactant or a label in the form of a component of a catalytic signal-producing system. This other affinity reactant will provide the conjugate with a reporter group in the form of an affinity tag, i.e. a group acting in a similar manner as a natural affinity group.

At the filing date variant (b) is of great interest for the invention. This variant includes that the analyte and its affinity counterpart (anti-analyte) are pre-incubated before reaching the porous bed, for instance outside the microfluidic device or in a separate mixing unit upstream the porous bed. The mixture is then transported through the porous bed where the free (=uncomplexed) anti-analyte (=analyte-related reactant) forms an affinity complex with an immobilized analyte analogue. In the case the analytically detectable group on the anti-analyte is an affinity group, then the anti-analyte captured on the porous bed may be detected by the use of an affinity reactant directed towards this group. This latter reactant then typically comprises a label in the form of a component of a catalytic signal-producing system. and is then used as the labeled measuring reagent. Alternatively the anti-analyte may comprise the component of the catalytic signal-producing system.

Competitive variants also include displacement assays in which an immobilized or immobilizable affinity complex that comprises two affinity counterparts (anti-analyte and analyte analogue) is incubated with a sample containing an analyte. Presuming the analyte analogue exhibits an analytically detectable group, displacement of the analyte analogue from the complex by the analyte will mean that the amount of detectable group in the complex is likely to change as a function of amount of analyte in the sample. In the case the detectable group in the analyte analogue is a component of the catalytic system, the analyte analogue may be used as the labeled measuring reagent of the invention. Alternatively, the analytically detectable group is a reporter group which may be detected by the aid of the labeled measuring reagent to be used in the invention, for instance a conjugate between an affinity reactant directed towards the detectable group and a component of a catalytic signal-producing system.

Competitive variants are particularly adapted for analytes that have difficulties in binding two or more affinity counterparts simultaneously, i.e. relatively small molecules.

Non-Competitive Protocols

These protocols typically utilize non-limiting amounts of one or more affinity counterparts to the analyte.

The most important non-competitive protocols are sandwich protocols which typically comprise formation of an immobilized or immobilizable complex in which an analyte is sandwiched between two affinity counterparts (anti-analytes). One of the counterparts is analytically detectable and the other immobilized or immobilizable. The analytically detectable anti-analyte may comprise the component of the catalytic signal-producing system. In other variants the detectable anti-analyte may comprise an affinity group (reporter group) that can be measured by the use of an affinity reactant that comprises a binding site for this group and also the component of the catalytic signal-producing system. This latter affinity reactant may thus be a conjugate between a) an affinity reactant that is a counterpart to the reporter group, and b) a component of the catalytic signal-producing system.

Another non-competitive variant utilizes only one affinity counterpart (anti-analyte) to the analyte in immobilized or immobilizable form (immobilized anti-analyte). In this case complex formation leads to an immobilized complex, or a soluble complex that subsequently is immobilized. In one variant the affinity counterpart which is immobilized or immobilizable has been labeled with an analytically detectable group that changes its activity when the analyte binds to the anti-analyte. The analytically detectable group may be of the same general kind as suggested above for competitive and/or sandwich protocols.

Non-competitive protocols have their greatest potential for molecules that are able to simultaneously bind two or more affinity counterparts, i.e. large molecules.

For non-competitive protocols it is in most instances preferred to form the complexes discussed above in immobilized form, i.e. by starting from an immobilized affinity reactant and then step-wise built the various complexes on the porous bed. Each step may comprise reaction between two, three, four or more affinity reactants. For competitive variants it is preferred to form a soluble complex and then capture the free uncomplexed anti-analyte on a porous bed comprising an immobilized analyte analogue.

Immobilizable reagents or complexes are typically immobilized after complex formation by affinity adsorption to the porous nl-beds used in the present invention.

Microfluidic Devices

A microfluidic device comprises one, two or more microchannel structures each of which is intended for carrying out the above-mentioned type of assay by transporting and processing one or more nl-aliquots of liquid containing the analyte and/or the necessary reagents for obtaining a labeled measuring reagent bound to a nl-bed. This does not exclude that larger volumes, such as in the interval 1-50 μl, and/or other liquids such as washing liquids may also be processed in a microfluidic device as long as at least one nl-aliquot is handled within the device.

A microchannel structure of a microfluidic device thus contains one or more cavities and/or conduits that have a cross-sectional dimension that is ≦10³ μm, preferably ≦5×10² μm, such as ≦10² μm. The nl-range has an upper limit of 5,000 nl. In most cases it relates to volumes ≦1,000 nl, such as ≦500 nl or ≦100 nl.

A microchannel structure typically comprises all the functional parts that are necessary for performing the intended assay within a microfluidic device, i.e. typically one, two, three or more functional parts selected among: a) inlet arrangements comprising for instance an inlet port/inlet opening, possibly together with a volume-metering unit, b) microconduits for liquid transport, c) reaction microcavities; d) mixing microcavities; e) units for separating particulate matters from liquids (may be present in the inlet arrangement), f) units for separating dissolved or suspended components in the sample from each other, for instance by capillary electrophoresis, chromatography and the like; g) detection microcavities; h) waste conduits/microcavities; i) valves; j) vents to ambient atmosphere; etc. A functional part may have more than one functionality, e.g. reaction microcavity and a detection microcavity may coincide. Various kinds of functional units in microfluidic devices have been described by Gyros AB/Amersham Pharmacia Biotech AB: WO 9955827, WO 9958245, WO 02074438, WO 0275312, WO 03018198, WO 03024598 and by Tecan/Gamera Biosciences: WO 0187487, WO 0187486, WO 0079285, WO 0078455, WO 0069560, WO 9807019, WO 9853311.

The microfluidic device may also comprise common microchannels/micro conduits connecting different microchannel structures. Common channels, such as common distribution manifold and common waste functions including their various parts such as inlet ports, outlet ports, vents, etc., are considered part of each of the microchannel structures they are communicating with.

Common microchannels make it possible to construe microfluidic devices in which the microchannel structures form networks. See for instance U.S. Pat. No. 6,479,299 (Caliper)

Each microchannel structure has at least one inlet opening for liquids and at least one outlet opening for excess of air (vents). Certain outlet vents may also function as outlets for waste and/or excess liquids.

The number of microchannel structures/device is typically ≦10, e.g. ≦25 or ≦90 or ≦180 or ≦270 or ≦360.

Different principles may be utilized for transporting the liquid within the microfluidic device/microchannel structures between two or more of the functional parts described above. Inertia force may be used, for instance by spinning the disc as discussed in the subsequent paragraph. Other useful forces are capillary forces, electrokinetic forces, non-electrokinetic forces such as capillary forces, hydrostatic pressure etc.

The microfluidic device typically is in the form of a disc. The preferred formats have an axis of symmetry (Cₙ) that is perpendicular to the disc plane, where n is an integer ≦2, 3, 4 or 5, preferably ∞ (C_{∞}): In other words the disc may be rectangular, such as in the form of a square, or have other polygonal forms. It may also be circular (C_{∞}). Once the proper disc format has been selected centrifugal force may be used for driving liquid flow, e.g. by spinning the device around a spin axis that typically is perpendicular or parallel to the disc plane. In the most obvious variants at the priority date, the spin axis coincides with the above-mentioned axis of symmetry. See the patent publications discussed above in the name of Gyros AB and Gamera Biosciences/Tecan. Preferred systems using spin axes that are not perpendicular to a disc plane are described in PCT/SE03/01850 (Gyros AB).

For preferred centrifugal-based variants, each microchannel structure comprises an upstream section that is at a shorter radial distance than a downstream section relative to the spin axis.

The preferred devices are typically disc-shaped with sizes and forms similar to the conventional CD-format, e.g. sizes that corresponds CD-radii that are the interval 10%-300% of the conventional CD-radii. The upper and/or lower sides of the disc may or may not be planar.

Microchannels/microcavities of a microfluidic device may be manufactured from an essentially planar substrate surface that exhibits the channels/cavities in uncovered form that in a subsequent step are covered by another essentially planar substrate (lid). See WO 9116966 (Pharmacia Biotech AB), WO 0154810 (Gyros AB), and WO 03055790 (Gyros AB). The material of the substrates may be selected among various kinds of inorganic and organic material, for instance polymeric material, such as plastics.

For aqueous liquids an essential part of the inner surfaces of the microchannel structures should have water contact angles ≦90°, such as ≦60° or ≦40° or ≦30° or ≦20° at the temperature of use or 25° C. At least two or three of the inner walls enclosing the channels should comply with this range. Surfaces in passive valves, anti-wicking means etc are excluded from these general rules. Surfaces made in plastics typically need to be hydrophilized. Useful hydrophilization protocols are for instance given in WO 9529203 (Pharmacia Biotech AB), WO 9800709 (Pharmacia Biotech AB, WO 0146637 (Gyros AB), WO 0056808 (Gyros AB) and WO 03086960 (Gyros AB) etc.

Non-wettable surface breaks (water contact angles ≧90°) may be introduced at predetermined positions in the inner walls of the microchannel structures before covering the uncovered microchannel structures (WO 9958245, Amersham Pharmacia Biotech AB) and WO 0185602, Πmic AB & Gyros AB). For aqueous liquids this means hydrophobic surface breaks. Surface breaks may be used for controlling the liquid flow within the structures, e.g. anti-wicking, passive valves, directing liquids etc.

Porous Beds

The porous bed is present in a reaction microcavity and typically comprises a capturing affinity reactant immobilized and homogeneously distributed in the bed. Several beds may be layered directly on top of each other and differ with respect to kind and/or concentration of capturing affinity reactant.

The porous bed is typically a) the inner surface of a porous monolith that wholly or partly will occupy the interior of the reaction microcavity, or b) a population of porous or non-porous particles that are packed to a porous bed.

A porous monolith may be fabricated in one piece of material or may comprise particles that are attached to each other.

By the term “porous particles” is meant that the particles can be penetrated by soluble reactants that are to be incorporated into the affinity complex. This typically means Kav values within the interval of 0.4-0.95 for at least one, preferably all, of these reactants. Non-porous particles have a Kav-value below 0.4 with respect to the same reactants. Porous monoliths have pores that are large enough to permit mass transport of the reactants through the matrix by the liquid flow applied.

The particles may be spherical or non-spherical. With respect to non-spherical particles, diameters and sizes refer to the “hydrodynamic” diameters.

The particles are preferably monodisperse (monosized) by which is meant that the population of particles placed in a reaction microcavity has a size distribution with more than 95% of the particles within the range of the mean particle size ±5%. Population of particles that are outside this range are polydisperse (polysized).

The porous bed may or may not be transparent for the principle used for measuring the complex.

The material in the porous bed, e.g. the particles, is typically polymeric, for instance a synthetic polymer or a biopolymer. The term biopolymer includes semi-synthetic polymers comprising a polymer chain derived from a native biopolymer. The particles and other forms of solid phases are typically hydrophilic in the case the liquid flow is aqueous. In this context hydrophilic encompasses that a porous solid phase, e.g. a packed bead, will be penetrated by water by self-suction. The term also indicates that the surfaces of the particles shall expose a plurality of polar functional groups in which there is a heteroatom selected amongst oxygen, sulphur, and nitrogen. Appropriate functional groups can be selected amongst hydroxy groups, straight eythylene oxide groups ([—CH₂CH₂O—]ₙ, n an integer >0, such as ≧2 or ≧3 or more), amino groups, carboxy groups, sulphone groups etc, with preference for those groups that are neutral independent of pH and/or are bound directly to a carbon atom, for instance sp³-hybridised. A hydrophobic particle or porous monolith may be hydrophilized, for instance by introducing hydrophilic groups. The coating and hydrophilization technique may be similar to the technique presented in WO 9529203 (Pharmacia Biotech AB), WO 9800709 (Pharmacia Biotech AB, Arvidsson & Ekström), WO 0146637 (Gyros AB), WO 0056808 (Gyros AB) and WO 03086960 (Gyros AB), for instance.

The techniques for immobilization of an affinity reactant to a solid phase may be selected amongst those that are commonly known in the field. Immobilization may thus be via covalent bonds, affinity bonds (for instance affinity bonds), physical adsorption (mainly hydrophobic interaction) etc. Examples of biospecific affinity bonds that can be used are bonds a) between streptavidin and a biotinylated affinity reactant, b) between high affinity antibody and a haptenylated affinity reactant etc, and vice versa. See for instance the experimental part.

Signal Data Treatment

In a particular preferred variant of the present invention the distribution of the measured signal across the surface of the porous bead viewed from above is used for calculating the true signal related to the analyte from the labeled measuring reagent. See for instance WO 03025548 (Gyros AB) and WO 03056517 (Gyros AB). In preferred variants one starts with obtaining a raw data image which subsequently is processed step-by-step by one or more different steps (methods) for reducing various kinds of noise contribution in the raw data image. Thus this processing may include one or more of the following steps:

Reducing background radiation (step α)

Reducing peak disturbances (step β)

Locating the detection area (the true surface area of the porous bed) within a larger search area comprising the detection area/determining a global treshold value (step χ)

Moving/removing binary artifacts (step δ)

Removing unwanted areas of the detection area (step ε)

Applying default detection area in noisy images (step φ)

Step α comprises two main variants. The first variant includes obtaining a background image prior to the formation of signal-emitting product formed by action of the signal-producing catalytic system. This background image is correlated to the raw data image obtained after formation of the signal-emitting product in the porous bed, and subsequently the value of the raw data signal for each pixel of the background image is deducted from the raw data signal of the corresponding pixel in the raw data image obtained after formation of the signal-emitting product. The background raw data image is preferably obtained from signal data collected as close as possible before formation of the signal-emitting product. In the second variant a median value of the background signal data is used for the deduction instead of a true background image. This median value can be obtained from a true background image or approximated from the signal raw data obtained after formation of the signal-emitting product.

Various details of steps α to φ are given in WO 03025548 (Gyros AB) and WO 03056517 (Gyros AB) which are hereby incorporated by reference.

Experimental Part

### Tyramid Signal Amplification

The protocol used was a non-competitive sandwich protocol utilizing a porous bed comprising immobilized strepavidin sensitized with biotinylated anti-analyte antibody. As detection antibody was used a different anti-analyte antibody tagged with a hapten (digoxigenin) combined with an anti-digoxigenin antibody labeled with a horseradish peroxidase. The substrate used contains a fluorophore that became immediately immobilized to the solid phase (Tyramid Signal Amplification kit) upon action of the peroxidase. As a reference method was used a variant in which the anti-analyte antibody tagged with hapten was replaced with the same anti-analyte antibody labeled with the same fluorophor as used in the Tyramid Signal Amplification kit. The reference method only comprised washing steps after the fluorescently labeled antibody had been captured on the porous bed.

The microfluidic device was in the form of a circular disc (CD) intended for using centrifugal force by spinning for driving liquid flow. The device was of the same general type as described in SE 0300822-4 (Gyros AB), Patent Application SE2004/000440 (Gyros AB). See also WO 02075312 (Gyros AB) and WO 03025548 (Gyros AB) in which similar structures also are given.

Chemicals

| | |
|---|---|
| Human Myoglobin | Human cardiac myoglobin (product no: 30-AM20) was purchased |
| | from Fitzgerald Industries International, Concord, MA) |
| Capturing reagent | Mouse monoclonal antibody (8E11.1) directed against human |
| | myoglobin (LabAs, Tartu, Estonia) that was labelled with EZ-Link |
| | Sulfo-NHS-LC-Biotin (Product no: 21335; Pierce, Rockford, IL) |
| | according to manufacturers instructions. |
| Detecting reagent | Mouse monoclonal antibody (2F9.1) directed against human |
| | myoglobin (LabAs, Tartu, Estonia) that was labelled with Alexa 647 |
| | (Product no: A-20186; Molecular Probes, Eugene, OR) according to |
| | manufacturers instructions. |
| Digoxigenin labelling | Mouse monoclonal antibody (2F9.1) directed against human |
| | myoglobin was labelled with the hapten digoxigenin using the DIG |
| | Protein Labelling kit (Product no: 1 367 200; Roche Molecular |
| | Biochemicals, Mannheim, Germany) according to manufacturers |
| | instructions |
| Digoxigenin detecting | Mouse monoclonal directed against digoxigenin and labelled with |
| reagent | Horse Radish Peroxidase (HRP) Products no: ab6212) was |
| | purchased from Abcam, Cambridge, UK. |
| Tyramid Signal | A kit containing all necessary reagents to perform the Tyramid |
| Amplification | Signal Amplification step (Product no: T-20916) was purchased |
| | from Molecular Probes, Eugene, OR |
| Wash buffer | 0.015 M Phosphate buffer, pH 7.4 containing 0.15 M NaCl and |
| | 0.01% Tween 20 |
| Phosphate buffer | 0.015 M Phosphate buffer, pH 7.4 containing 0.15 M NaCl (PBS) |
| Assay buffer | PBS + 1% BSA |

A standard curve was constructed using human cardiac myoglobin diluted in 1% BSA (Calbiochem) covering a range between 12.5 pM to 12.5 nM.

The biotinylated capturing reagent was diluted in assay buffer to 0.2 mg/ml

The Alexa 647 labelled detecting reagent was diluted in assay buffer to 50 nM concentration

The digoxigenin labelled monoclonal antibody was diluted to 50 nM concentration in assay buffer

The HRP anti-digoxigenin antibody was diluted in the range of 1:10 to 1:100 in assay buffer

The Alexa 647 labelled reactive tyramid compound was used in dilutions 1:25 to 1:200

Process

CDs containing 112 identical microstructures, each of them being pre-packed with a column of 10-15 nl containing streptavidin coupled beads of 15 □m size, were used in the process.

200 nl aliquots of liquids were volume defined in the CD either via individual or common inlet and processed as described in the sequence below. Each reaction step was performed under constant liquid flow over the column for 2-4 min, 50-100 nl/min. Washing between reaction steps was performed under higher flow rates.

Fluorescence measurements was carried out as outlined in WO 03025548 (Gyros AB) with noise reduction by utilizing the principles various kinds of background images as of outlined in PCT/SE02/002455 (Gyros AB).

Process steps

| Reference method | TSA method |
|---|---|
| Wash 1 | Wash 1 |
| Addition of biotinylated capturing | Addition of biotinylated capturing |
| antibody at 0.2 mg/ml | antibody at 0.2 mg/ml |
| Wash 2 | Wash 2 |
| Addition of analyte | Addition of analyte |
| Wash 3 | Wash 3 |
| Wash 4 | Wash 4 |
| Addition of fluorescence-labelled | Addition of Digoxigenin-labelled |
| detecting antibody (anti-myoglobin) | second antibody (anti-myoglobin) |
| Wash 5 | Wash 5 |
| Wash 6 | Wash 6 |
| Wash 7 | Addition of HRP labelled anti- |
| | digoxigenin antibody |
| Wash 8 | Wash 7 |
| Wash 9 | Wash 8 |
| | Addition of substrate |
| | Wash 9 |
| | Wash 10 |
| | Wash 11 |
| | Wash 12 |
| | Wash 13 |

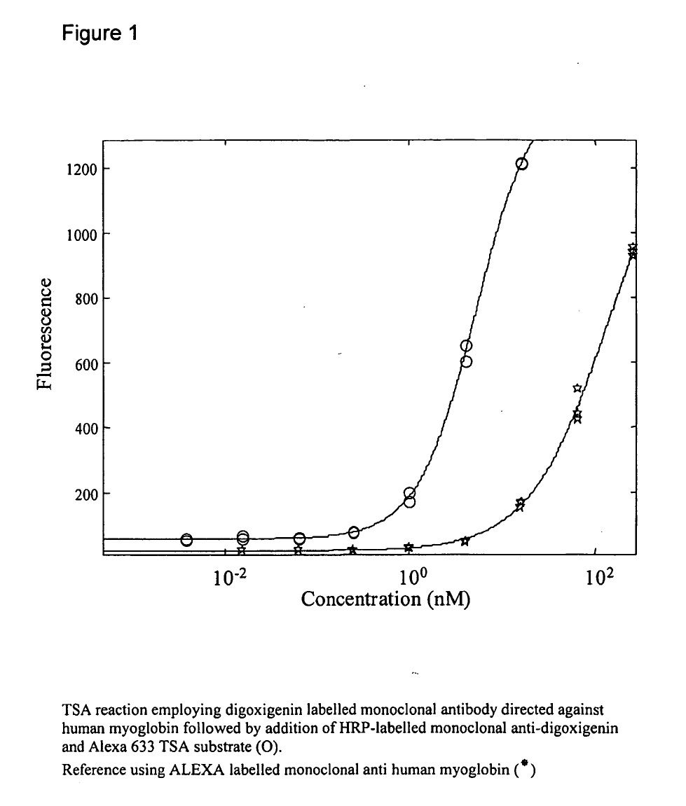

The reference curves for the reference method and the innovative variant given above is shown in FIG. 1. A significant increase in sensitivity is noted.

1. A method for measuring the signal from a label in a labeled measuring reagent that has been specifically adsorbed to its affinity counterpart in a zone of a porous nanolitre (nl) bed that is present in a microchannel structure of a microfluidic device, said measuring being part of an assay for determining an analyte of a sample by performing one or more heterogeneous specific affinity reactions which comprises that said labeled measuring reagent becomes affinity bound to said zone, wherein a) at least one of the heterogeneous affinity reactions comprises specifically adsorbing an analyte-related reactant with an excessive amount of its counterpart immobilized and homogeneously distributed within the porous nl-bed, and b) said labeled measuring reagent comprises a label that is a component of a catalytic signal-producing system that converts a substrate to an immobilized analytically detectable product, and the measuring comprises the steps of:
i. providing the components of said catalytic signal-producing system that are necessary for the formation of said immobilized product within said zone, and
ii. producing said immobilized analytically detectable product within said zone.

2. The method of claim 1, wherein the formation of said product is taking place under static conditions and/or flow conditions.

3. The method of claim 2, wherein the formation is taking place in two or more substeps with a fresh aliquot of said components displacing the preceding aliquot.

4. The method of claim 1, wherein the catalytic signal-producing system comprises an enzyme system and the label is selected from the group consisting of enzyme, cofactor, and co-enzyme.

5. The method of claim 1, wherein said product is a precipitate.

6. The method of claim 1, wherein said product is covalently linked to said porous bed by direct covalent bonds to the matrix of the porous bed and/or to an affinity reactants.

7. The method of claim 1, wherein said product is immobilized via specific affinity adsorption to said porous bed.

8. The method of claim 1, wherein said assay has a competitive format.

9. The method of claim 8, wherein said porous bed comprises immobilized analyte analogue and one of said heterogeneous affinity reaction comprises affinity adsorption of an anti-analyte to said porous bed preferably under flow conditions.

10. The method of claim 8, wherein said labeled measuring reagent is said anti-analyte labeled with said component or an affinity reactant directed towards a binding site on said anti-analyte which is not interfering with the affinity reaction between the analyte and the anti-analyte, preferably with
a) said anti-analyte being a conjugate between a) an unconjugated anti-analyte and b) a reporter group, and
b) said labeled measuring reagent being a conjugate between a) an affinity reactant directed towards said reporter group, and b) said component.

11. The method of claim 1, wherein said assay has a non-competitive format.

12. The method of claim 11, wherein said porous bed comprises an immobilized anti-analyte, that one of said heterogeneous affinity reactions comprises affinity adsorption of said analyte to said immobilized anti-analyte, preferably under flow conditions.

13. The method of claim 11, wherein said non-competitive format is a sandwich format.

14. The method of claim 11, wherein said labeled measuring reagent is a conjugate between a) an anti-analyte that is directed to a different binding site and b) said component.

15. The method of claim 14, wherein said non-competitive format is a sandwich format comprising formation of the complex:
anti-analyte(1)—analyte—anti-analyte (2)
in which
a) anti-analyte(1) is directly or indirectly immobilized to said porous bed and
b) anti-analyte(2) is
i) said labeled measuring reagent, or
ii) an anti-analyte that comprises an analytically detectable group which can be detected by the use of a form of said labeled measuring reagent which is directed towards said group.

16. The method of claim 1, wherein said microfluidic device comprises a plurality of said microchannel structures and porous beds.

17. The method of claim 1, wherein said device provides for common flow control of liquid transport in said microchannel structures.

18. The method of claim 1, wherein said component is a non-substrate component of said catalytic signal-producing system.
